(19) United States
(12) Patent Application Publication
(10) Pub. No.: US 2021/0186831 A1
(43) Pub. Date: Jun. 24, 2021

(54) THERAPEUTIC COMPOSITIONS AND METHODS USING EXOSOMES DERIVED FROM HUMAN DERMAL FIBROBLASTS

(71) Applicant: North Carolina State University, Raleigh, NC (US)

(72) Inventors: Ke Cheng, Raleigh, NC (US); Shiqi Hu, Raleigh, NC (US)

(21) Appl. No.: 17/126,572

(22) Filed: Dec. 18, 2020

Related U.S. Application Data
(60) Provisional application No. 62/950,653, filed on Dec. 19, 2019.

(51) Int. Cl. A61K 8/14; C12N 5/00; C12N 5/077

(52) U.S. Cl. CPC A61K 8/14; C12N 5/0656; C12N 5/0062

(57) ABSTRACT

The present disclosure provides compositions and methods relating to the use of exosomes derived from human dermal fibroblasts (HDFs). In particular, the present disclosure provides novel compositions and methods for generating and maintaining exosomes derived from HDF spheroids, as well as compositions and methods for delivering the exosomes to a subject for various therapeutic purposes, such as the treatment of skin conditions.

### CROSS REFERENCE TO RELATED APPLICATIONS

This application claims priority to and the benefit of U.S. Provisional Patent Application No. 62/950,653 filed Dec. 19, 2019, which is incorporated herein by reference in its entirety for all purposes.

### GOVERNMENT SUPPORT

This invention was made with government support under grant numbers HL123920, HL137093, HL144002, and HL146153 awarded by the National Institutes of Health. The government has certain rights in the invention.

### FIELD

The present disclosure provides compositions and methods relating to the use of exosomes derived from human dermal fibroblasts (HDFs). In particular, the present disclosure provides novel compositions and methods for generating and maintaining exosomes derived from HDF spheroids, as well as compositions and methods for delivering the exosomes to a subject for various therapeutic purposes, such as the treatment of skin conditions.

### BACKGROUND

Various materials, such as antioxidants, retinoids, peptides, and growth factors have been used to protect or repair the skin. Additionally, dermal fillers have proven more effective at smoothing facial contours and much longer-lasting than many topical treatments, and autologous patient fat or collagen are relatively safe filler materials. Unfortunately, the methods and treatments currently available for treating the skin have significant drawbacks. For example, autologous patient fat or collagen is tedious to harvest and easily resorbed by the body within several months. Although autologous dermal fibroblast injections are capable of improving facial contour defects and creating a continuous protein repair system (12-48 months) to reduce wrinkle formation, fibroblasts gradually lose their capacity to proliferate and synthesize collagen with aging. Additionally, both intrinsic and extrinsic aging change the quantity and proliferation rates of dermal fibroblasts, reduce collagen production, and accelerate the degradation of dermal matrix by matrix-degrading metalloproteinases (MMPs), thereby inducing wrinkles.

Exosomes have recently received much scientific attention since they can mediate cell-to-cell communication and regulate the properties of target cells. For example, exosomes derived from human induced pluripotent stem cells (iPSCs) were reported to significantly reduce the expression level of MMPs and senescence-associated beta-galactosidase (SA-β-Gal), and upregulate the expression of collagen in HDFs. Topical treatments with exosomes could be applied to the epidermis and be absorbed through the skin; however, the efficiency is largely limited due to poor penetration through the stratum. Therefore, there is a need for alternative therapeutic approaches and delivery methods for treating skin conditions.

### SUMMARY

Embodiments of the present disclosure include a composition comprising a plurality of exosomes derived from human dermal fibroblast (HDF) spheroids. In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p.

In some embodiments, the plurality of exosomes are derived from HDF spheroids cultured using three-dimensional (3D) cell culture. In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p, as compared to a naturally occurring HDF-derived exosome.

In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p, as compared to an HDF-derived exosome cultured using two-dimensional (2D) cell culture.

Embodiments of the present disclosure also include a method of generating a plurality of exosomes capable of modulating at least one characteristic of skin tissue. In some embodiments, the method includes culturing human dermal fibroblast (HDF) spheroids using three-dimensional (3D) cell culture; and isolating a plurality of exosomes from the HDF spheroids.

In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p.

In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p, as compared to a naturally occurring HDF-derived exosome.

In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p, as compared to an HDF-derived exosome cultured using two-dimensional (2D) cell culture.

Embodiments of the present disclosure also include a method of treating a skin condition or disease. In some embodiments, the method comprises administering a plurality of exosomes derived from human dermal fibroblast (HDF) spheroids to a subject in need thereof. In some embodiments, administering the plurality of exosomes modulates at least one characteristic of the subject's skin tissue.

In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p.

In some embodiments, the plurality of exosomes are derived from HDF spheroids cultured using three-dimensional (3D) cell culture. In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p, as compared to a naturally occurring HDF-derived exosome.

In some embodiments, the plurality of exosomes have at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p, as compared to an HDF-derived exosome cultured using two-dimensional (2D) cell culture.

In some embodiments, the plurality of exosomes are administered to the subject's skin via injection, microinjection (microneedles), intradermal (ID) injection, subcutaneous (SC) injection, a non-invasive method, needle-free injection, or topical application.

In some embodiments, modulating at least one characteristic of the subject's skin tissue comprises at least one of: (i) increasing expression of Transforming Growth Factor-β1 (TGF-β1); (ii) increasing expression of procollagen type I; (iii) decreasing expression of Tumor Necrosis Factor-α (TNF-α); (iv) decreasing expression of Matrix Metallopeptidase 1 (MMP1); (v) decreasing expression of Matrix Metallopeptidase 9 (MMP9); and/or (vi) increased dermal collagen deposition.

In some embodiments, modulating at least one of: (i) increasing expression of Transforming Growth Factor-β1 (TGF-β1); (ii) increasing expression of procollagen type I; (iii) decreasing expression of Tumor Necrosis Factor-α (TNF-α); (iv) decreasing expression of Matrix Metallopeptidase 1 (MMP1); (v) decreasing expression of Matrix Metallopeptidase 9 (MMP9); and/or (vi) increased dermal collagen deposition, treats the subject's skin condition or disease.

In some embodiments, the skin condition or disease comprises cutaneous aging. In some embodiments, the skin condition or disease comprises cutaneous photoaging. In some embodiments, the skin condition or disease comprises chronological aging.

### BRIEF DESCRIPTION OF THE DRAWINGS

FIGS. 1A-1D: Comparison of 2D HDFs and 3D spheroids. A) Photographs of human dermal fibroblasts (HDFs) and spheroids formed (scale bar:100 μm). B) Evaluation of vimentin (green channel) and CD34 (red channel) expression in 2D and 3D cultured HDFs (P6). DAPI (blue) was used to locate the nuclei of the cells (scale bar: 40 μm). C) Schematic illustration of the culture process. D) Pro-collagen I expression in 2D HDFs and spheroids from passage 2, 4, 6 and after UVB exposure. Expression assessed by ELISA. n=5, *p<0.05, **p<0.01, ****p<0.0001.

FIGS. 2A-2C: Comparison of exosomes derived from Mesenchymal Stromal Cells (MSCs), 2D and 3D HDFs. A) Cytokine array of 2D HDF-XOs and 3D HDF-XOs (P6) by densitometric analysis (n=3). B) Heatmap of fibrosis related miRNA array incubated with 2D HDF-XOs, 3D spheroids-XOs and MSC-XOs (n=3). In 3D HDF-XOs, hsa-miR-196a-5p and hsa-miR-744-5p were downregulated compared to 2D HDF-XOs, while hsa-miR-133a-3p, hsa-miR-223-3p, hsa-5011-5p, hsa-miR-325, hsa-miR-199b-5p and hsa-miR-34a-5p were upregulated compared to both MSC-XOs and 2D HDF-XOs. C) miRNAs of 2D HDF-XOs, 3D HDF-XOs and MSC-XOs expressed at relatively high levels. n=3, *p<0.05, **p<0.01.

FIGS. 3A-3B: The effects of exosomes on HDFs. A) Wound recovery rates of HDFs, modeled by cell scratch assays. B) The scratch closure rate is presented over time (n=3). C) HDF proliferation with the treatment of different exosomes, n=3, n.s. means no significant difference, *p<0.05, **p<0.01, ***p<0.001, ****p<0.0001.

FIGS. 4A-4E: Comparison of intradermal injection with a syringe to needle-free injection with a jet injector. A) Schematic illustration of needle injection and needle-free injection. B) Comparison of the syringe and jet injector properties and applications. C) Exosomes were labeled with DiD and injected into the dorsal skin of a nude mouse. D) The mice were sacrificed 12 h after injection. Skin slices from the left and right sites were imaged via Confocal microscopy. Highly concentrated exosomes (red) accumulated between the dermis and hypodermis in the mice that were intradermally injection. In mice treated with the jet injector, the exosomes dispersed well in both the dermis and hypodermis. E) Representative skin histology. Scale bar: 100 μm.

FIGS. 5A-5B: Effect of Retinoic acid (RA) and exosomes from different cells on wrinkle formation in UVB-irradiated nude mice. A) Microscopic observation of replicas (scale bar: 100 μm) and B) photographs of dorsal skin of mice from different groups (n=3).

FIGS. 6A-6D: Histological analysis of the dorsal surface of treated and untreated nude mice after UVB irradiation. A) Masson's trichrome staining. From left to right: Sham, saline, dermal application of 0.05% retinoic acid (every other day), PRP, MSC-XOs/PRP and 3D spheroids XOs/PRP (last three received one-time injections); scale bar: 290 μm. B) Corresponding H&E staining, scale bar: 290 μm. C) Epidermal and D) dermal thickness analysis. n=9 (3 mice per group, 3 spots analyzed for each sample), n.s. means no significant difference, *p<0.05, **p<0.01, ***p<0.001.

FIGS. 7A-7G: Anti-photoaging mechanism signaling pathway analysis. A) Western blot of dorsal skin of different groups. B-F) Quantification of procollagen 1, MMP1, TGF-β, TNF-α and IL-1β (n=3), n.s. means no significant difference, *p<0.05, **p<0.01, ***p<0.001, ****p<0.0001. G) Schematic illustration of the mechanism of 3D HDF-XOs treatment.

FIGS. 8A-8B: A) Comparison of growth factors secreted by HDFs (P1) and MSC by B) densitometric analysis, n=3. **p<0.01, ***p<0.001, ****p<0.0001.

FIGS. 9A-9C: A) Size measurement of exosomes using DLS, n=3. B) Western blot showing Alix and CD81 blotting on exosome lysate. C) Transmission electron microscopy (TEM) image of 3D HDF-XOs.

FIGS. 10A-10B: The effects of 3D HDF-XOs on the ki67 expression of UVB-irradiated HDFs, n=5. ***p<0.001.

FIG. 11: The effects of exosomes on senescent phenotype of UVB-irradiated HDFs by SA-β-Gal staining, n=3. Scale bar: 200 μn.

FIG. 12: Cytokine analysis of skin samples from the control group and the 3D HDF-XOs treated group, n=3. *p<0.05, **p<0.01, ***p<0.001.

FIG. 13: Western blot showing Procollagen type I, MMP1, TGF-β, TNF-α, IL-1β and GAPDH from the skin lysis of different groups. n=3.

FIG. 14: Mice body weight changes during treatment. n=3, n.s. means no significant difference.

FIG. 15: IVIS imaging of DiR-labeled exosomes injected via DERMOJET.

FIG. 16: Distribution of exosomes delivered by DERMOJET in porcine skin. 50 μL DiR labeled exosomes were injected to the abdominal skin of Yorkshire piglets via DERMOJET. Then the skin was harvested and sectioned. From left to right are DiR labeled exosomes (red), DAPI (blue) and overlay.

FIGS. 17A-17B: A) MiRNA array raw data of 3D HDF-XOs VS. MSC-XOs; and B) 3D HDF-XOs VS. 2D-HDF-XOs.

### DETAILED DESCRIPTION

Embodiments of the present disclosure provide novel compositions and methods for generating and maintaining exosomes derived from HDF spheroids. As described further herein, three-dimensional (3D) culture of human dermal fibroblast (HDF) spheroids were developed to stimulate the expression of a selected group of cytokines and regain the collagen synthesizing ability of photoaged HDFs. To induce photoaging in vitro and in vivo, dermal fibroblasts and nude mice were irradiated with ultraviolet B light (UVB, 311 nm). In addition, associated signal pathways were analyzed to identify the biological processes underlying the specific alterations of proteins and miRNA cargos in exosomes.

Both extrinsic and intrinsic aging can lead to a microenvironment with enhanced oxidative stress and inflammatory levels, as well as senescent dermal fibroblasts. During the aging process, the upregulation of MMP production and the downregulation of collagen production lead to age-related skin disorders, including weakened dermal structure and poor mechanical integrity. Results of the present disclosure indicate that spheroid formation can restore the function of aged HDFs. In addition, the results provided herein indicate that 3D HDF-XOs regulate dermal fibroblasts to induce efficient collagen biosynthesis and ameliorate inflammation in the skin caused by UVB irradiation. Thicker dermal matrix was successfully achieved in nude mice using needle free injection of 3D HDF-XOs. In accordance with these data, results of miRNA profiling data provided herein indicate that the down regulation of miR-196a, as well as the upregulation of miR-133a and miR-223, contribute to the process. 3D HDF-XOs inhibited UVB-induced MMP1 expression, restored procollagen type1 and activated TGF-β signal pathway. In addition, 3D HDF-XOs ameliorated the inflammation and senescence of the skin through the down regulating TNF-α.

Thus, as described further herein, delivery of exosomes (e.g., with a jet injector) provides an effective approach for transdermal delivery of exosomes for therapeutic purposes. 3D HDF-XOs were more effective than MSC-XOs at regulating dermal fibroblast proliferation, migration and protein expression, thus reducing skin-aging.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like.

The term “cell culture process” or “cell culture” generally refers to the process by which cells are grown or maintained under controlled conditions. The cell culture process may take place in vitro or ex vivo. In some embodiments, a cell culture process has both an expansion phase and a production phase. In some embodiments, the expansion and production phases are separated by a transition or shift phase. “Culturing” a cell refers to contacting a cell with a cell culture medium under conditions suitable to for growing or maintaining the cell. A “cell culture” can also refer to a solution containing cells. In some embodiments, cell cultures can be three-dimensional (3D) or two-dimensional (2D). Generally, 2D cell culture systems grow cells on flat dishes, typically made of plastic. The cells are put onto coated surfaces where they adhere and spread in a two-dimensional fashion. Generally, 3D cell culture systems can be described as the culture of living cells within micro-assembled devices and supports that provide a three-dimensional structure mimicking tissue and organ specific microarchitecture (see, e.g., John W. Haycock et al. “3D Cell Culture: A Review of Current Approaches and Techniques”).

The terms “medium” and “cell culture medium” (plural, “media”) generally refer to a nutrient source used for growing or maintaining cells. As is understood by a person of ordinary skill in the art based on the present disclosure, a growth medium or cell culture medium is a liquid or gel designed to support the growth of microorganisms, cells, or small plants. Cell culture media generally comprise an appropriate source of energy and compounds which regulate the cell cycle. A typical culture medium can be composed of, but not limited to, a complement of amino acids, vitamins, inorganic salts, glucose, and serum as a source of growth factors, hormones, and attachment factors. In addition to nutrients, the medium also helps maintain pH and osmolality.

The terms “administration of” and “administering” a composition as used herein refers to providing a composition of the present disclosure to a subject in need of treatment. The compositions of the present disclosure may be administered by topical (e.g., in contact with skin or surface of body cavity), oral, parenteral (e.g., intramuscular, intraperitoneal, intravenous, ICV, intracisternal injection or infusion, subcutaneous injection, or implant), by spray, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing, e.g., exosomes and/or miRNAs of the present disclosure and a pharmaceutically acceptable carrier and/or excipient. When exosomes and/or miRNAs of the present disclosure are used contemporaneously with one or more other drugs, a pharmaceutical composition containing such other drugs in addition to the exosomes and/or miRNAs of the present disclosure are contemplated. Accordingly, the pharmaceutical compositions of the present disclosure include those that also contain one or more other active ingredients, in addition to a exosomes and/or miRNAs of the present disclosure. The weight ratio of the exosomes and/or miRNAs of the present disclosure may be varied and will depend upon the effective dose of each ingredient. Generally, an effective dose of each will be used. Combinations of exosomes and/or miRNAs of the present disclosure and other active ingredients will generally also be within the aforementioned range, but in each case, an effective dose of each active ingredient should be used. In such combinations the exosomes and/or miRNAs of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition, and/or to improve/enhance one or more aspects of a subject's physical health. The compositions can be formulated according to known methods for preparing pharmaceutically useful compositions (e.g., exosome preparation). Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “effective amount” generally means that amount of a drug or pharmaceutical agent that will elicit the biological or medical response of a tissue, system, animal or human that is being sought, for instance, by a researcher or clinician. Furthermore, the term “therapeutically effective amount” generally means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “combination” and derivatives thereof, as used herein, generally means either, simultaneous administration or any manner of separate sequential administration of a therapeutically effective amount of Compound A, or a pharmaceutically acceptable salt thereof, and Compound B or a pharmaceutically acceptable salt thereof, in the same composition or different compositions. If the administration is not simultaneous, the compounds are administered in a close time proximity to each other. Furthermore, it does not matter if the compounds are administered in the same dosage form (e.g., one compound may be administered topically and the other compound may be administered orally).

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, cell biology, genetics and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Compositions and Methods

The present disclosure provides compositions and methods relating to the use of exosomes derived from human dermal fibroblasts (HDFs). In particular, the present disclosure provides novel compositions and methods for generating and maintaining exosomes derived from HDF spheroids, as well as compositions and methods for delivering the exosomes to a subject for various therapeutic purposes, such as the treatment of skin conditions.

In accordance with these embodiments, the present disclosure includes a composition comprising a plurality of exosomes derived from human dermal fibroblast (HDF) spheroids. In some embodiments, the plurality of exosomes have been engineered to include at least one of: (i) increased expression of Tissue Inhibitor of Metalloproteinases-1 (TIMP-1); (ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one of the following miRNAs: hsa-miR-196a-5p and/or hsa-miR-744-5p.

In some embodiments, the plurality of exosomes have been engineered to include increased expression of Tissue Inhibitor of Metalloproteinases-1 (TIMP-1). In some embodiments, the plurality of exosomes have been engineered to include increased expression and/or activity of Tissue Inhibitor of Metalloproteinases-1 (TIMP-1).

In some embodiments, the plurality of exosomes have been engineered to include increased expression of at least one of the following miRNAs: hsa-miR-133a-3p, hsa-miR-223-3p, hsa-5011-5p, hsa-miR-325, hsa-miR-199b-5p, and/or hsa-miR-34a-5p. In some embodiments, the plurality of exosomes have been engineered to include increased expression of at least two of the following miRNAs: hsa-miR-133a-3p, hsa-miR-223-3p, hsa-5011-5p, hsa-miR-325, hsa-miR-199b-5p, and/or hsa-miR-34a-5p. In some embodiments, the plurality of exosomes have been engineered to include increased expression of at least three of the following miRNAs: hsa-miR-133a-3p, hsa-miR-223-3p, hsa-5011-5p, hsa-miR-325, hsa-miR-199b-5p, and/or hsa-miR-34a-5p. In some embodiments, the plurality of exosomes have been engineered to include increased expression of at least four of the following miRNAs: hsa-miR-133a-3p, hsa-miR-223-3p, hsa-5011-5p, hsa-miR-325, hsa-miR-199b-5p, and/or hsa-miR-34a-5p. In some embodiments, the plurality of exosomes have been engineered to include increased expression of at least five of the following miRNAs: hsa-miR-133a-3p, hsa-miR-223-3p, hsa-5011-5p, hsa-miR-325, hsa-miR-199b-5p, and/or hsa-miR-34a-5p. In some embodiments, the plurality of exosomes have been engineered to include increased expression of all six of the following miRNAs: hsa-miR-133a-3p, hsa-miR-223-3p, hsa-5011-5p, hsa-miR-325, hsa-miR-199b-5p, and/or hsa-miR-34a-5p. In some embodiments, the plurality of exosomes have been engineered to include decreased expression of at least one of the following miRNAs: hsa-miR-196a-5p and/or hsa-miR-744-5p. In some embodiments, the plurality of exosomes have been engineered to include decreased expression of both of the following miRNAs: hsa-miR-196a-5p and/or hsa-miR-744-5p. As would be understood by one of ordinary skill in the art based on the present disclosure a plurality of exosomes can be engineered to include increased and/or decreased expression of any combination of the aforementioned miRNAs.

In accordance with these embodiments, the plurality of exosomes can be derived from HDF spheroids cultured using three-dimensional (3D) cell culture. In some embodiments, the plurality of exosomes have at least one of: (i) increased expression of Tissue Inhibitor of Metalloproteinases-1 (TIMP-1); (ii) increased expression of at least one (and up to all six) of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one (or both) of the following miRNAs: hsa-miR-196a-5p and/or hsa-miR-744-5p, as compared to a naturally occurring HDF-derived exosome. As would be understood by one of ordinary skill in the art based on the present disclosure a plurality of exosomes can be engineered to include increased and/or decreased expression of any combination of the aforementioned miRNAs.

In some embodiments, the plurality of exosomes have at least one of: (i) increased expression of Tissue Inhibitor of Metalloproteinases-1 (TIMP-1); (ii) increased expression of at least one (and up to all six) of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one (or both) of the following miRNAs: hsa-miR-196a-5p and/or hsa-miR-744-5p, as compared to an HDF-derived exosome cultured using two-dimensional (2D) cell culture. As would be understood by one of ordinary skill in the art based on the present disclosure a plurality of exosomes can be engineered to include increased and/or decreased expression of any combination of the aforementioned miRNAs.

Embodiments of the present disclosure also include a method of generating a plurality of exosomes capable of modulating at least one characteristic of skin tissue. In some embodiments, the method includes culturing human dermal fibroblast (HDF) spheroids using three-dimensional (3D) cell culture; and isolating a plurality of exosomes from the HDF spheroids.

In some embodiments, the plurality of exosomes have at least one of: (i) increased expression of Tissue Inhibitor of Metalloproteinases-1 (TIMP-1); (ii) increased expression of at least one (and up to all six) of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one (or both) of the following miRNAs: hsa-miR-196a-5p and/or hsa-miR-744-5p. As would be understood by one of ordinary skill in the art based on the present disclosure a plurality of exosomes can be engineered to include increased and/or decreased expression of any combination of the aforementioned miRNAs.

In some embodiments, the plurality of exosomes have at least one of: (i) increased expression of Tissue Inhibitor of Metalloproteinases-1 (TIMP-1); (ii) increased expression of at least one (and up to all six) of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one (or both) of the following miRNAs: hsa-miR-196a-5p and/or hsa-miR-744-5p, as compared to a naturally occurring HDF-derived exosome. As would be understood by one of ordinary skill in the art based on the present disclosure a plurality of exosomes can be engineered to include increased and/or decreased expression of any combination of the aforementioned miRNAs.

In some embodiments, the plurality of exosomes have at least one of: (i) increased expression of Tissue Inhibitor of Metalloproteinases-1 (TIMP-1); (ii) increased expression of at least one (and up to all six) of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one (or both) of the following miRNAs: hsa-miR-196a-5p and/or hsa-miR-744-5p, as compared to an HDF-derived exosome cultured using two-dimensional (2D) cell culture. As would be understood by one of ordinary skill in the art based on the present disclosure a plurality of exosomes can be engineered to include increased and/or decreased expression of any combination of the aforementioned miRNAs.

Embodiments of the present disclosure also include a method of treating a skin condition or disease. In some embodiments, the method comprises administering a plurality of exosomes derived from human dermal fibroblast (HDF) spheroids to a subject in need thereof. In some embodiments, administering the plurality of exosomes modulates at least one characteristic of the subject's skin tissue.

In some embodiments, the plurality of exosomes have at least one of: (i) increased expression of Tissue Inhibitor of Metalloproteinases-1 (TIMP-1); (ii) increased expression of at least one (and up to all six) of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one (or both) of the following miRNAs: hsa-miR-196a-5p and/or hsa-miR-744-5p. As would be understood by one of ordinary skill in the art based on the present disclosure a plurality of exosomes can be engineered to include increased and/or decreased expression of any combination of the aforementioned miRNAs.

In some embodiments, the plurality of exosomes are derived from HDF spheroids cultured using three-dimensional (3D) cell culture. In some embodiments, the plurality of exosomes have at least one of: (i) increased expression of Tissue Inhibitor of Metalloproteinases-1 (TIMP-1); (ii) increased expression of at least one (and up to all six) of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one (or both) of the following miRNAs: hsa-miR-196a-5p and/or hsa-miR-744-5p, as compared to a naturally occurring HDF-derived exosome. As would be understood by one of ordinary skill in the art based on the present disclosure a plurality of exosomes can be engineered to include increased and/or decreased expression of any combination of the aforementioned miRNAs.

In some embodiments, the plurality of exosomes have at least one of: (i) increased expression of Tissue Inhibitor of Metalloproteinases-1 (TIMP-1); (ii) increased expression of at least one (and up to all six) of the following miRNAs: hsa-miR-133a-3p, hsa-miR-223-3p, hsa-5011-5p, hsa-miR-325, hsa-miR-199b-5p, and/or hsa-miR-34a-5p; and/or (iii) decreased expression of at least one (or both) of the following miRNAs: hsa-miR-196a-5p and/or hsa-miR-744-5p, as compared to an HDF-derived exosome cultured using two-dimensional (2D) cell culture. As would be understood by one of ordinary skill in the art based on the present disclosure a plurality of exosomes can be engineered to include increased and/or decreased expression of any combination of the aforementioned miRNAs.

In some embodiments, the plurality of exosomes are administered to the subject's skin via injection, microinjection (microneedles), intradermal (ID) injection, subcutaneous (SC) injection, a non-invasive method, needle-free injection, or topical application.

In some embodiments, modulating at least one characteristic of the subject's skin tissue comprises at least one of: (i) increasing expression of Transforming Growth Factor-β1 (TGF-β1); (ii) increasing expression of procollagen type I; (iii) decreasing expression of Tumor Necrosis Factor-α (TNF-α); (iv) decreasing expression of Matrix Metallopeptidase 1 (MMP1); (v) decreasing expression of Matrix Metallopeptidase 9 (MMP9); and/or (vi) increased dermal collagen deposition. In some embodiments, modulating at least one of: (i) increasing expression of Transforming Growth Factor-β1 (TGF-β1); (ii) increasing expression of procollagen type I; (iii) decreasing expression of Tumor Necrosis Factor-α (TNF-α); (iv) decreasing expression of Matrix Metallopeptidase 1 (MMP1); (v) decreasing expression of Matrix Metallopeptidase 9 (MMP9); and/or (vi) increased dermal collagen deposition, treats the subject's skin condition or disease. In some embodiments, the skin condition or disease comprises cutaneous aging. In some embodiments, the skin condition or disease comprises cutaneous photoaging. In some embodiments, the skin condition or disease comprises chronological aging.

Embodiments of the present disclosure also include a kit comprising any of the exosomes and/or miRNAs described herein, and at least one container and/or administration device. In some embodiments, the kit further includes instructions for administering the composition to a human, including such information as dosing regimens, frequency of administration, routes of administration, side effects, and the like. In some embodiments, the kit includes a device that can be used to administer any of the compositions described herein, including but not limited to, a syringe, an injector, an applicator, a depressor, and the like, to an individual in need thereof.

### 3. Materials and Methods

Cells and Exosomes. Normal human dermal fibroblasts (HDFs, PCS201012) (ATCC, Manassas, USA) were cultured in Dulbecco's Modified Eagle's Medium (DMEM) supplemented with a Low Serum Growth Supplement kit (S00310) (ThermoFisher Scientific, Rockford, Ill., USA) in cell incubator. Bone Marrow-Derived Mesenchymal Stem Cells (Normal, Human, ATCC® PCS-500-012™) were in mesenchymal stem cell basal medium (ATCC-PCS®-500-030™) with growth kit (ATCC-PCS®-500-041™). Spheroids were formed by transfer HDFs (one million cells) into ultra-low attachment flasks (Corning™ 3815, 75 cm2). Then, conditioned medium was collected for exosome isolation. Briefly, for monolayer cells, cells were seeded into T-175 flasks. Once the cells were 80% confluent, they were washed with phosphate-buffered saline (PBS) three times. Then, 20 mL serum-free DMEM (for HDFs) or mesenchymal stem cell basal medium (for MSCs) was added to each T-175 flask for additional five days. For HDF spheroids, the medium was replaced with serum-free DMEM on day 3 after spheroids formation. Then, the conditioned medium was collected after five days. For both monolayer cells and spheroids, after the incubation with serum-free DMEM Conditioned medium was collected and filtered through 0.22-μm filter unit (SCGP00525) (Sigma-Aldrich, St. Louis, Mo.) to get rid of cells and debris. To remove the proteins and concentrate exosomes in the medium, the medium was then centrifuged and washed with PBS through ultra-15 centrifugal filter unit (100 KDa, UFC910024) (MilliporeSigma, Burlington, Mass., USA). The exosomes were concentrated to 1010/mL and stored at −80° C. for further use.

Characterization. The concentration of exosomes was examined by a NanoSight LM10 (Malvern Instruments Ltd., UK). Size of exosomes was measured by dynamic light scattering (DLS, Malvern ZEN 3600 Zetasizer). The morphology of exosomes was recorded using a transmission electron microscope (TEM, JEOL JEM-2000FX). Total RNA of exosomes was extracted using the RNeasy Mini kit (Qiagen, Germany) and the miRNA analysis was performed with an miScript miRNA PCR Array Human Fibrosis kit (Qiagen, Germany).

Cytokine Array. The comparison of HDF-CM and MSC-CM was performed using Human Cytokines Array (AAH-CYT-1000) (RayBiotech, Peachtree Corners, Ga.) according to the manufacturer's instruction. The cytokines of 2D HDF and 3D HDF were analyzed using a Human Angiogenesis Array C1000 (RayBiotech, AAH-ANG-1000). Skin samples were analyzed using a Mouse Apoptosis Signaling Pathway Array (RayBiotech, AAM-APOSIG-1).

Ultraviolet B (UVB) Irradiation of HDFs. For in vitro experiments, HDFs were washed with PBS before the exposure to UVB. UVB dose (Philip, 311 nm, 20W/01, Germany) was 0.05 J/cm2/day for three days. Then, cells were incubated with serum-free DMEM with or without exosomes at 108/mL for another 24 h.

Procollagen Type 1 ELISA. Type 1 procollagen concentration was measured by ELISA (Abcam, Cambridge, UK). The absorbance at 450 nm was measured using a microplate spectrophotometer (BioTek Instruments, Winooski, Vt., USA).

Wound Healing Assay. The HDFs were seeded in two-well ibidi inserts (Minitube Canada, Ingersoll, Ontario) at 1×105 cells per well and cultured overnight to form a confluent monolayer at 37° C. in 5% CO2. The inserts were then carefully removed by peeling them back from one corner, and the cells were washed with serum-free DMEM once and then maintained in serum-free DMEM with or without exosomes (108/mL). At 0, 24 and 48 h, the scratch areas were imaged and measured using NIH ImageJ (n=3).

Proliferation Assay. HDF proliferation experiments were performed using a Cell Counting Kit-8 (96992-500TESTS-F, Sigma-Aldrich). Briefly, the HDFs were seeded at 5000 cells per well in a 96-well plate and settled down overnight. Then, the medium was replaced with serum-free DMEM with or without exosomes. After another 48 h, CCK-8 reagent was added to each well and incubated for 1 h. Absorbance at 450 nm was read and recorded using a microplate spectrophotometer.

Animal Studies. All animal work was compliant with the Institutional Animal Care and Use Committee (IACUC) of North Carolina State University. For the topical experiment, the nude mouse dorsal skin (6-8 weeks old, The Jackson Laboratories) was irradiated with UVB every other day for 8 weeks. The irradiation intensity represented as the minimal erythemal dose (MED), was set at 1 MED during the first 2 weeks (60 mJ/cm2), and was elevated to 2 MED (120 mJ/cm2) in the 3rd week, to 3 MED (180 mJ/cm2) in the 4th week and to 4 MED (240 mJ/cm2) during the 5th-8th weeks of the experiment. The total irradiated UVB volume was approximately 80 MED. Treatment: eighteen nude mice were randomly divided into six groups of three mice each: (a) no UVB exposure (Sham); (b) UVB irradiation alone (Control); (c) UVB irradiation with 0.05% retinoic acid (RA); (d) UVB irradiation with 2D HDF-XOs; (e) UVB irradiation with 3D HDF-XOs and (f) UVB irradiation with MSC-XOs. In this experiment, 0.05% RA was used as a positive control and applied on the dorsal skin every other day. Exosomes were all delivered by Dermo-jet Model G (DJ-05, Robbins Instruments, USA). Dermo-jet exosome delivery consisted of one-time injections in ten different sites evenly on the whole dorsal skin. The exosome dose used was 1010/mL and 1 mL per mouse. For each mouse, the whole back skin can be divided into at least three parts to be analyzed (i.e. used three samples for each group). Results were consistent among groups with no significant differences within groups.

Skin Replica. Replica SILFLO and Ring Locator were brought from Clinical & Derm, Dallas, Tex., USA. Skin replica was performed at the end of the treatment on the back skin of mice. Then, the replica was observed under a stereo microscope (Olympus SZX7) and corresponding images were analyzed by ImageJ (NIH).

Statistical Analysis. The experimental data provided herein were presented as mean±standard deviation. Comparisons among more than two groups were performed using one-way ANOVA followed by post-hoc Bonferroni test. Single, double, triple and quadra asterisks represent p<0.05, 0.01, 0.001 and 0.0001 respectively. All analysis was performed using GraphPad Prism 7 software (San Diego, Calif., USA).

Immunohistochemistry Assessment. Monolayer cells or spheroids were fixed with 4% paraformaldehyde (20 min at room temperature), blocked with Protein Block Solution (DAKO) containing 0.1% saponin (1 h at room temperature) and then incubated with primary antibodies diluted in the blocking solution overnight at 4° C. After washing with PBS, samples were stained with fluorescent secondary antibodies (1 h at room temperature). After washing with PBS again, slides were mounted with ProLong™ Diamond Antifade Mountant with DAPI (P36962, Thermo Fisher Scientific) and imaged with a Zeiss LSM 710 confocal microscope. Primary antibodies used: CD34 (ab81289, Abcam) and vimentin (ab8978, Abcam). Secondary antibodies used: goat anti-rabbit IgG-Alexa Fluor 594 conjugate (1:400, ab150080, Abcam) and goat anti-mouse IgG-Alexa Fluor 488 conjugate (1:400, ab150113, Abcam).

Cellular Senescence Assay. SA-beta-gal activity was assessed using a SA-beta-gal staining kit (Cell Signaling Technology, Boston, Mass., USA). HDFs with different treatment were fixed and stained at 37° C. overnight in freshly prepared SA-beta-gal staining solution.

Histological Analysis. Dorsal skin specimens of nude mice were obtained and fixed in 4% paraformaldehyde for at least 24 h. Next, they were embedded in paraffin and sectioned at 5 μm thicknesses. Briefly, H&E staining was conducted by deparaffinization, hydration, hematoxylin staining, eosin staining, and dehydration. To carry out the Masson's trichrome staining, the paraffin-embedded skin specimens were stained with Bouin's solution and Weigert's iron hematoxylin working solution, phosphomolybdic-phosphotungstic acid solution, aniline blue solution and dehydrated in series.

Western Blot Analysis. For western blot analysis, skin tissues were lysed by T-PER™ Tissue Protein Extraction Reagent (Fisher) and centrifuged at 12,000×g for 20 min at 4° C. Skin lysates were then homogenized to yield equivalent amounts of protein based on protein concentration measurements carried out with BCA protein assay kit (Thermo Scientific). Samples were electrophoresed through sodium dodecyl sulfate-polyacrylamide gel (SDS-PAGE), transferred to a nitrocellulose membrane (Amersham Pharmacia Biotech, Buckinghamshire, UK), blocked with 5% milk for 1 h under room temperature conditions, and primary antibodies were used for incubation with the membrane overnight at 4° C. The membrane was then washed three times and incubated with secondary antibody for 1 h at room temperature.

Primary antibodies used in the present disclosure: anti-Pro-Collagen Type 1, A1/COL1A1, (ABT257) (Sigma-Aldrich), MMP 1 (ab137332, Abcam), TNF-α (ab8348, Abcam), IL-1β (TE271712, Invitrogen), TGF-β (ab29769, Abcam), GAPDH (ab9835, Abcam). Secondary antibodies used: Goat anti-Rabbit IgG (H+L) Secondary Antibody, HRP (65-6120) (Invitrogen, Carlsbad, Calif.) and Goat anti-Mouse IgG (H+L) Secondary Antibody, HRP (31430, Invitrogen).

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

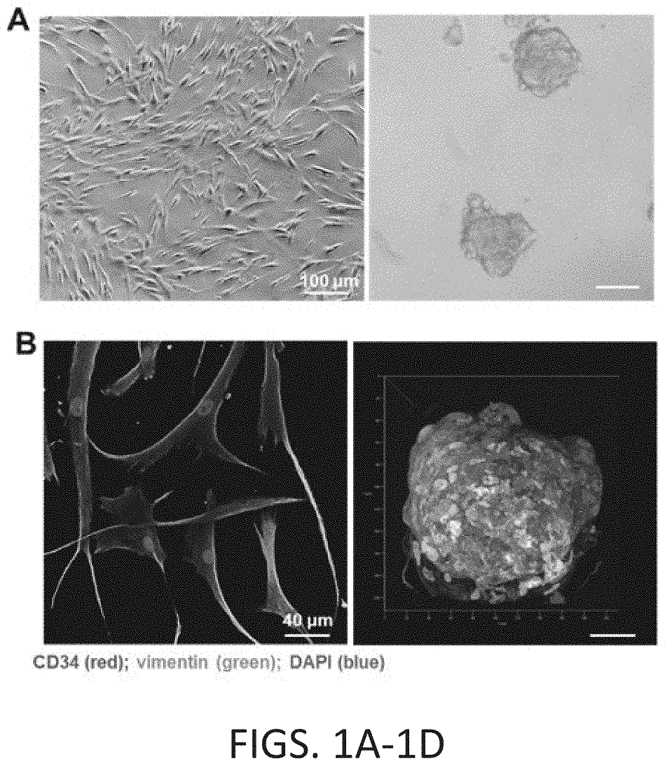

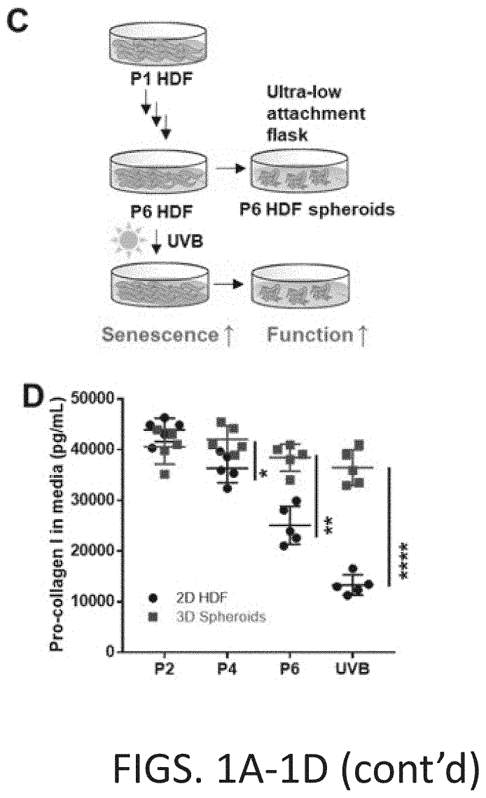

Comparison of Monolayer HDFs and Spheroids. Dermal fibroblasts are predominant mesenchymal cell type for extracellular matrix deposition and remodeling. Intrinsic and extrinsic aging, however, greatly reduce the ability of HDFs to proliferate and generate collagen, which in turn accelerates the breakdown of connective tissue and generates wrinkles. In the present disclosure, HDFs were obtained commercially and expanded. HDF spheroids were formed by passaging HDFs into ultra-low attachment tissue culture flasks. As shown in FIG. 1A, the morphology of HDFs in monolayer is a classic spindle-like shape. The spheroids are compact aggregates of HDFs cultured in suspension. They had diameters of 100-200 μm and had higher expressions of the proteins, vimentin and CD34 (FIG. 1B). Vimentin expression is highly related to fibroblast growth and collagen accumulation, while CD34^{pos} fibroblasts exhibited enhanced in vitro invasion and migration, which means that these cells retain, even enhance their proliferative ability when cultured in 3D spheroids. As illustrated in FIG. 1C, along with intrinsic aging (passage) and extrinsic aging (UVB) on 2D HDFs, partial cells were cultured into ultra-low attachment flasks to form corresponding 3D HDFs. Besides vimentin and CD34, the secretions from cells growing in suspended spheroids were differed greatly from those of monolayer cultured cells. For example, VEGF expression in HDFs was reported 22-fold higher in the three-dimensional culture system.

In the present disclosure, type I procollagen expression was detected using enzyme-linked immunosorbent assay (ELISA). Type I procollagen is a precursor to Type 1 collagen, which is the major structural protein in skin connective tissue. An emerging body of evidence suggests that dermal fibroblasts gradually loss their ability to produce type I procollagen by oxidative metabolism from UV irradiation and constant passaging driven senescence. FIG. 1D shows that after passaging and UVB irradiation, the procollagen type I synthesis ability of 2D HDFs was greatly suppressed. However, spheroids were able to restore the procollagen type 1 production of HDFs. Therefore, 3D culture was used as a potential approach to investigate skin aging.

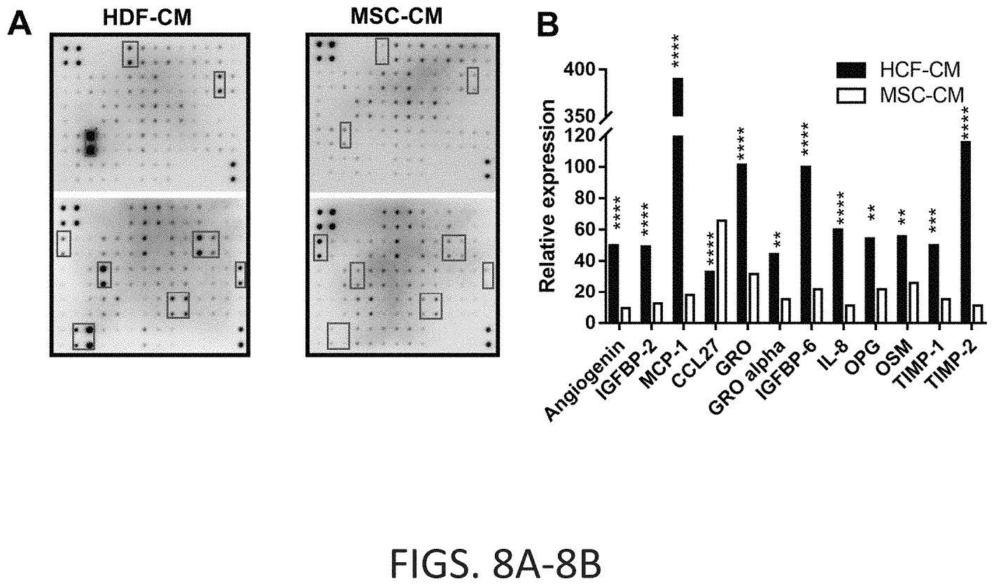

The secretomes from different cells were analyzed to explore possible components that might be effective against aging. Recently, many studies have examined the effects of stem cell-derived conditioned medium in wound healing and ischemic injury, mainly because they can suppress inflammation and promote angiogenesis. Conditioned medium from bone marrow-derived mesenchymal stem cells (BMMSC) was demonstrated to markedly reduce UV-induced MMP1 expression and increase pro-collagen synthesis. Thus, stem cell conditioned medium may have anti-aging agents that can be used to rejuvenate aged skin. In the present disclosure, to find out if 3D spheroids can regain the sternness of dermal fibroblasts and if the overexpressed factors or miRNAs are similar to stem cells, MSCs were used as the comparison. Growth factors secreted by fresh HDFs and MSCs were compared. As shown in FIG. 8, stem cells produce a series of growth factors, such as vascular endothelial growth factor (VEGF), epidermal growth factor (EGF), insulin-like growth factor binding protein (IGFBP), and basic fibroblast growth factor (bFGF). These cytokines have been shown to contribute to angiogenesis and injury repairment. Compared to MSCs, HDFs produce growth factors that are more related to collagen synthesis and dermal matrix remodeling, such as TIMP1 and TIMP2, which act as inhibitors of metalloproteinases (MMPs). As evidenced by the conditioned medium contents, dermal fibroblasts may be better able to regulate skin tissue compared to MSCs.

### Example 2

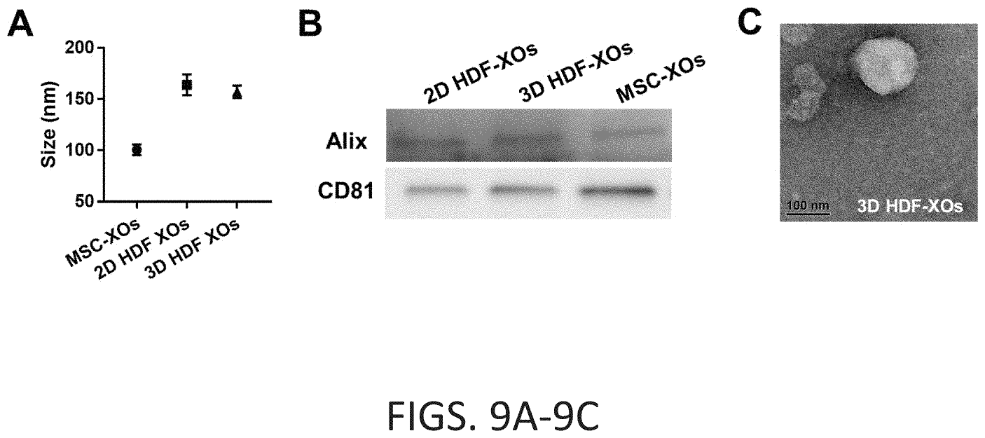

Characterization of Exosomes from 2D HDFs, 3D HDFs and MSCs. Studies have demonstrated that exosomes derived from conditioned medium are rich in various miRNAs and proteins which mediate the intercellular communication and the functions of HDFs, including the proliferation, collagen production and DNA repair. Exosomes from the conditioned medium of 2D HDFs, 3D HDFs, and MSCs were isolated to examine their effect on HDFs. Exosomes were characterized in terms of size distribution, zeta potential and surface marker expressions (FIG. 9). The mean particle diameters of MSC-XOs, 2D HDF XOs, and 3D HDF XOs were 97 nm, 162 nm, and 151 nm, respectively. All exosomes were positive for EV markers tetraspanins (CD81) and multivesicular body synthesis proteins (Alix). Analysis of miRNA cargo and proteomics was subsequently conducted.

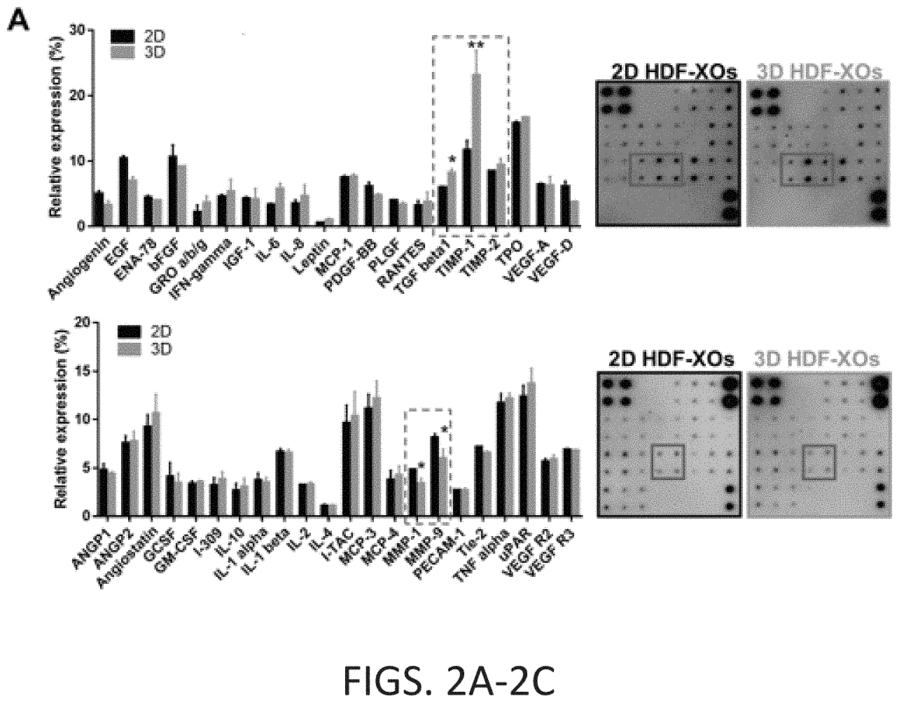

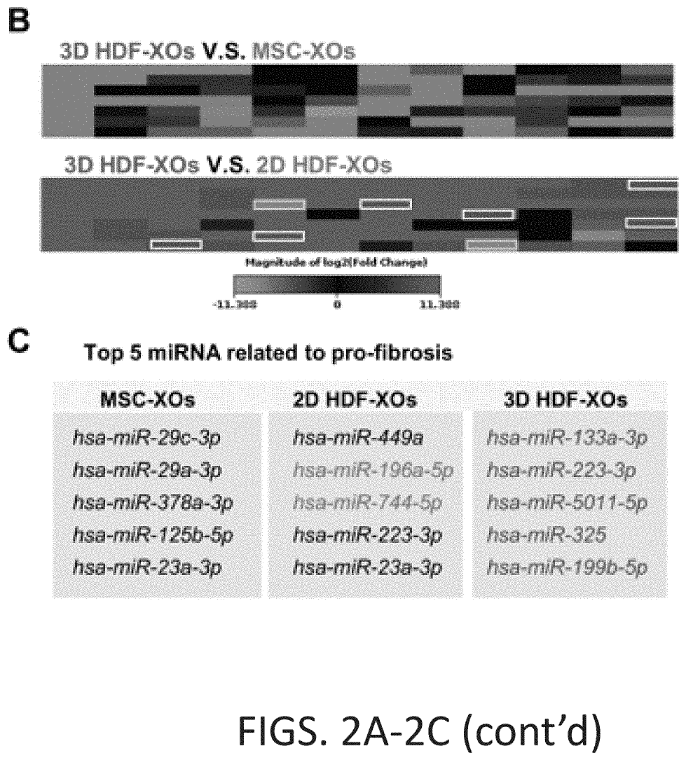

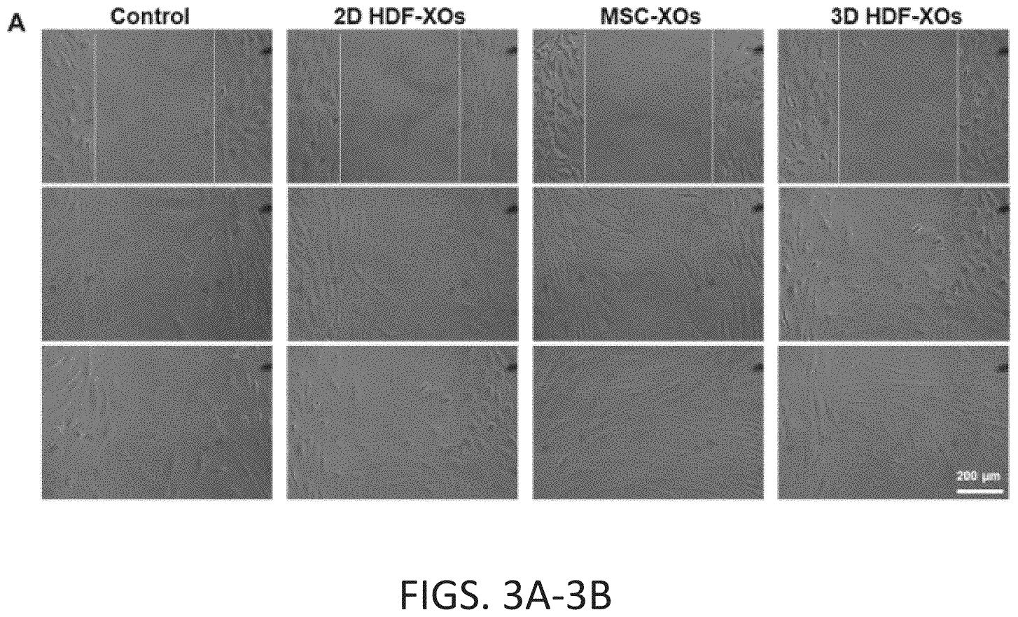

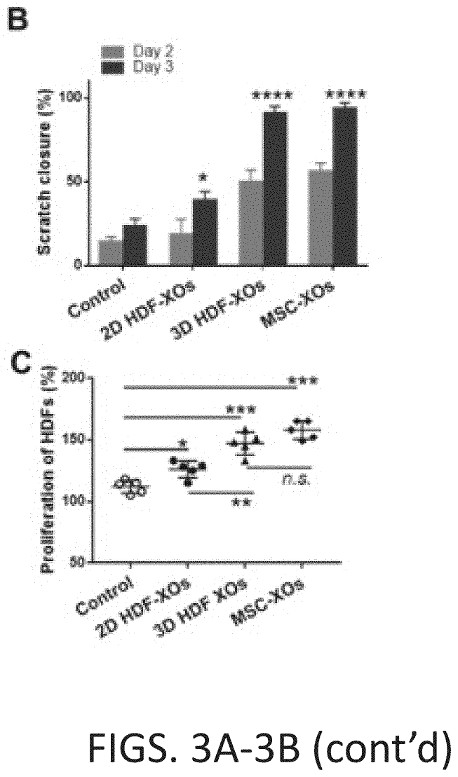

Cytokine arrays (FIG. 2A) showed that the most significantly enhanced protein in 3D HDF-XOs was TIMP1, which aids in the maintenance of collagen fibers. In addition, TGF-β1 was overexpressed, MMP1 and MMP9 were downregulated compared to 2D HDF-XOs. There was no significant difference for other proteins. The miRNAs of exosomes from MSCs have been explored. They have been involved in the Wnt signaling pathway, TGFβ signaling pathway and mitogen-activated protein kinase (MAPK) signaling pathway in wound healing process and anti-skin aging. In the pro-fibrosis related miRNA array of the present disclosure, MSC-XOs showed a higher expression of miRNAs from the miRNA29 family. The miRNAs in this family have been identified as potential posttranscriptional regulators of collagen genes. Moreover, these miRNAs are downstream of most of the profibrotic molecules produced, such as TGF-β. In 3D HDF-XOs, hsa-miR-196a-5p and hsa-miR-744-5p were downregulated compared to 2D HDF-XOs while hsa-miR-133a-3p, hsa-miR-223-3p, hsa-5011-5p, hsa-miR-325, hsa-miR-199b-5p and hsa-miR-34a-5p were upregulated compared to both MSC-XOs and 2D HDF-XOs (FIG. 2B). Experiments were conducted to investigate whether 3D cell culture would regain the stemness of dermal fibroblasts by upregulating miRNAs found in MSCs.

Results provided in the present disclosure indicated that 2D HDF-XOs were more similar to exosomes from MSCs. This suggests that 3D cell culture was able to upregulate different miRNAs that are important for tissue repair. Downregulated miR-196a would lead to a high expression of type I collagen in dermal fibroblasts. MiR-223 mimics were reported to alter the levels of different cytokines in the supernatant of cultured macrophages, such as enhancing IL-8 and IL-10 expression, and decreasing TNFα levels. Upregulated miR-133a was reported to downregulate pro-inflammatory cytokines. These results demonstrate that these exosomes may affect skin tissue through different signaling pathways and deliver different messages to dermal fibroblasts and other cells, such as keratinocytes and macrophages.

### Example 3

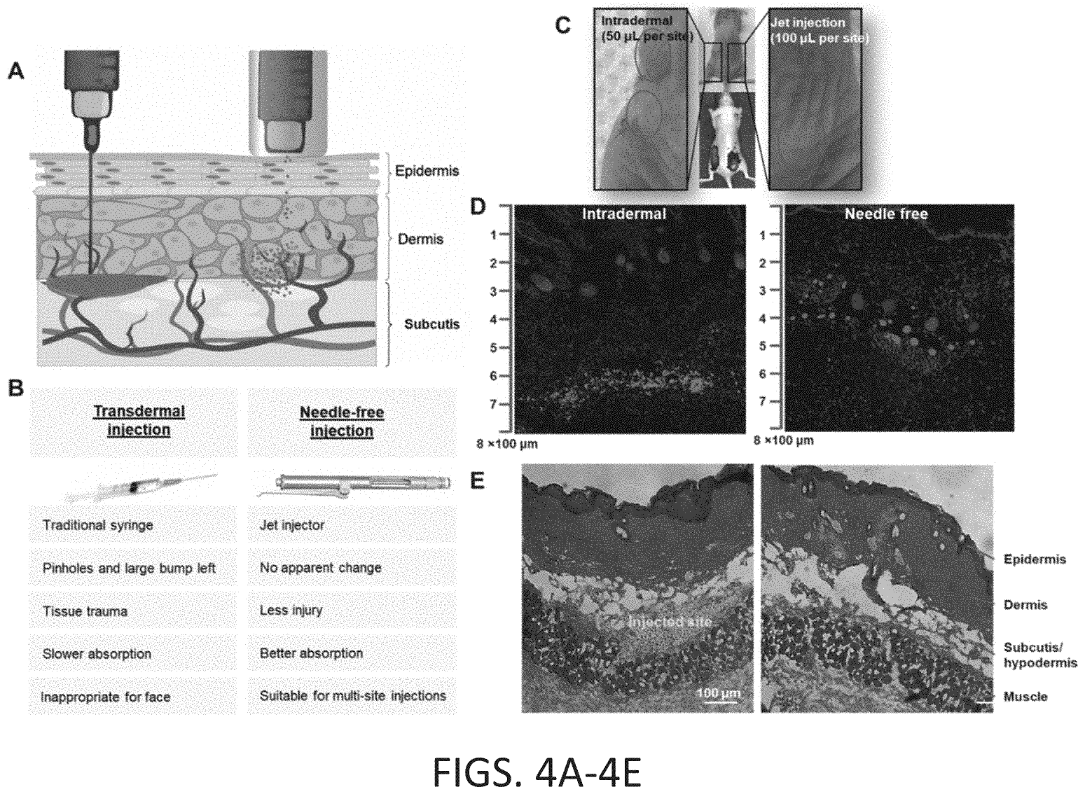

Effect of Different Exosomes on the Proliferation and Migration of HDFs. The effects of exosomes from three different types of cells on the proliferation, migration and function of HDFs in vitro were investigated. FIGS. 4A-4E show a comparison of intradermal injection with syringe to needle-free injection with jet injector. Exosomes were labeled with DiD and injected into the dorsal skin of a nude mouse. The intradermal injections were administered on the left side of the back (FIG. 4C; arrows indicate the injection sites). The jet injections were administered on the right side of the back. There was no obvious administration injury. A relatively large amount of solution pools in the tissue as a result of needle injections, which leads to local tissue trauma; however, jet injections result in wider penetration and better absorption.

The migration of HDFs were investigated with a wound healing assay (FIGS. 4A-4B). Compared to the control group (serum-free medium), all treated groups showed a significantly faster rate of wound recovery. At day 3, 3D HDF-XOs and MSC-XOs showed a full recovery, while 2D HDF-XOs showed a relative slower migration. The assessment of their effect on HDF proliferation was performed by a CCK8 assay. As shown in FIG. 4C, similar to the wound healing assay, both 3D HDF-XOs and MSC-XOs significantly promoted the proliferation of HDFs compared to the control group and 2D HDF XOs. Then, UVB irradiation was used to further induce the senescent phenotype of HDFs.

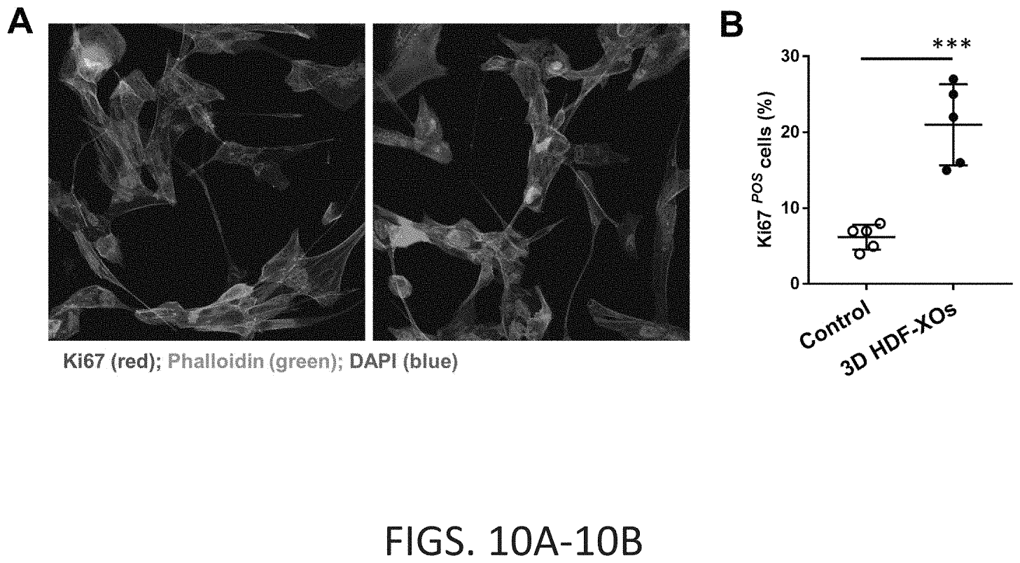

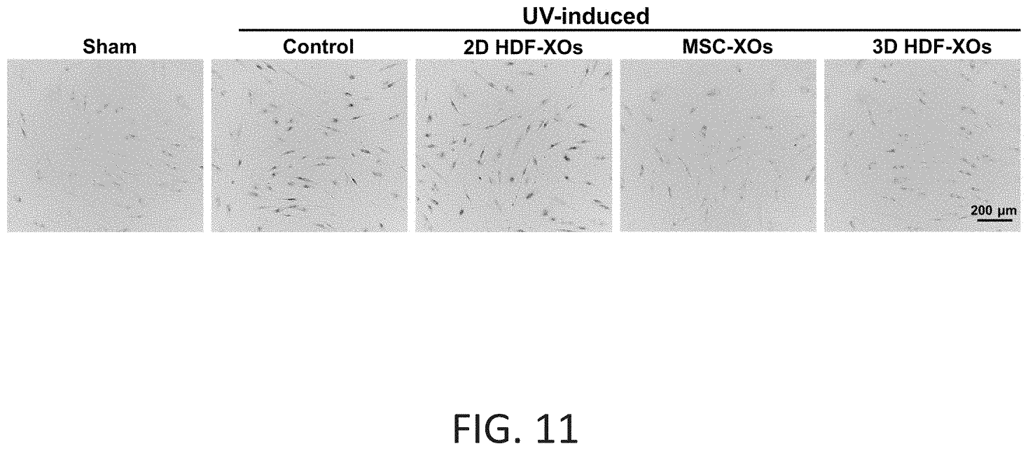

As shown in FIGS. 10A-10B, there were limited Ki67^{pos} HDFs after the irradiation. However, after incubation with 3D HDF-XOs, the expression of Ki67 was significantly increased. Senescence-associated beta-galactosidase (SA-β-gal) was also used to directly stain the senescent HDFs after UVB irradiation. As shown in FIG. 11, UVB irradiation induced a severe senescent phenotype of HDFs (bluish color) in HDFs. 2D HDF-XOs cannot reverse the senescence process, however, the addition of 3D HDF-XOs or MSC-XOs significantly prevented the induction of the senescent phenotype.

Collectively, these results demonstrate that 3D culture reversed the signs of photoaging and achieved similar results to MSC-XOs in vitro. These results suggest that the 3D culture of HDF spheroids is a vital step to regain the functional characteristics of passaged fibroblasts, and that exosomes derived from dermal fibroblast spheroids mediate the process.

### Example 4

Evaluation of Exosome Delivery with A Needle-Free Injector. To effectively regulate skin tissue, exosomes penetrate through the epidermis to reach the dermis. While topical treatments are the most common approach used to relieve skin aging, the efficiency of these treatments is poor due to lack of sufficient penetration into the deep dermis. Transdermal injections using syringes lead to bumps and local tissue trauma. Needle free injection technology has already highly benefited mass immunization programs due to it can bypass possible needle stick injuries, reusability and avoid needle phobia. Herein, the efficacy of a commercially available jet injector (e.g., needle-free injector) that pneumatically accelerates exosome solution into the dermis of skin (FIG. 4A) was evaluated. FIG. 4B summarized the advantages of needle-free injectors over traditional syringes, such as less injury and pain, better penetration and absorption, and more suitable for cosmetic usage. 3D HDF-XOs were labeled with DiD to facilitate the detection of their distribution in the dermis. The dispersion of exosomes through the histological analysis of skin biopsies was examined (FIGS. 4C-4E). The injection of a concentrated mass of exosomes with the syringe lead to accumulation. Compared to syringe injections, jet injector caused only invisible microtrauma to the dermis, which was beneficial since microtrauma would trigger natural wound healing processes and augment collagen generation therein. As shown in FIG. 4E, a mass of inflammatory cells will migrate and group to areas treated with needle injections, but jet injections induce no visible injuries.

### Example 5

Effects of Exosome Application in UVB-Induced Skin Photoaging in Nude Mice. Next, the efficacy of exosome treatment on reversing wrinkles in a nude mice model was evaluated. Repeated exposure to UV radiation injures the stratum corneum that accelerating ageing and increasing the risk of skin cancer, produces reactive oxygen species that upregulating MMPs and proinflammatory cytokines that damage the dermis, thereby leading to photoaging. After 8 weeks of UVB exposure (every other day), mice were divided into five treatment groups: control, 0.05% retinoic acid (RA, positive control), 2D HDF-XOs, 3D HDF-XOs and MSC-XOs. After the treatment, the body weight of mice was recorded every other day. Obvious better skin contour of 3D HDF-XOs treated group was observed started from week 3, so the state of the skin using skin replica, H&E, and Masson Trichrome staining was assessed on week 4.

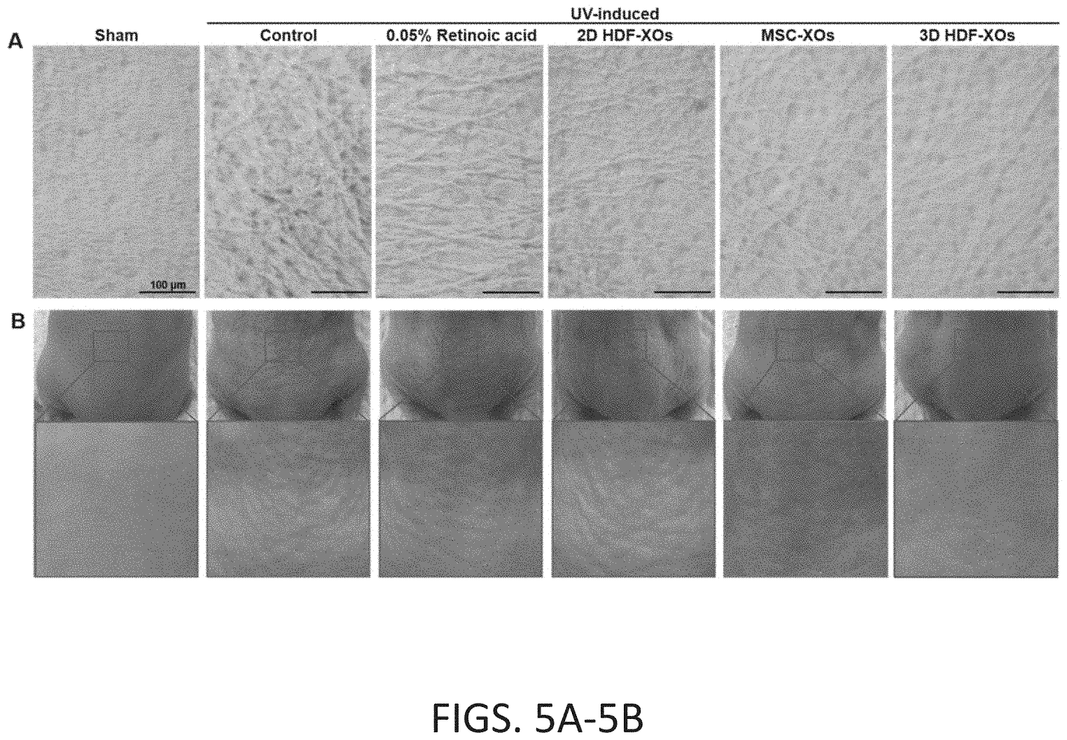

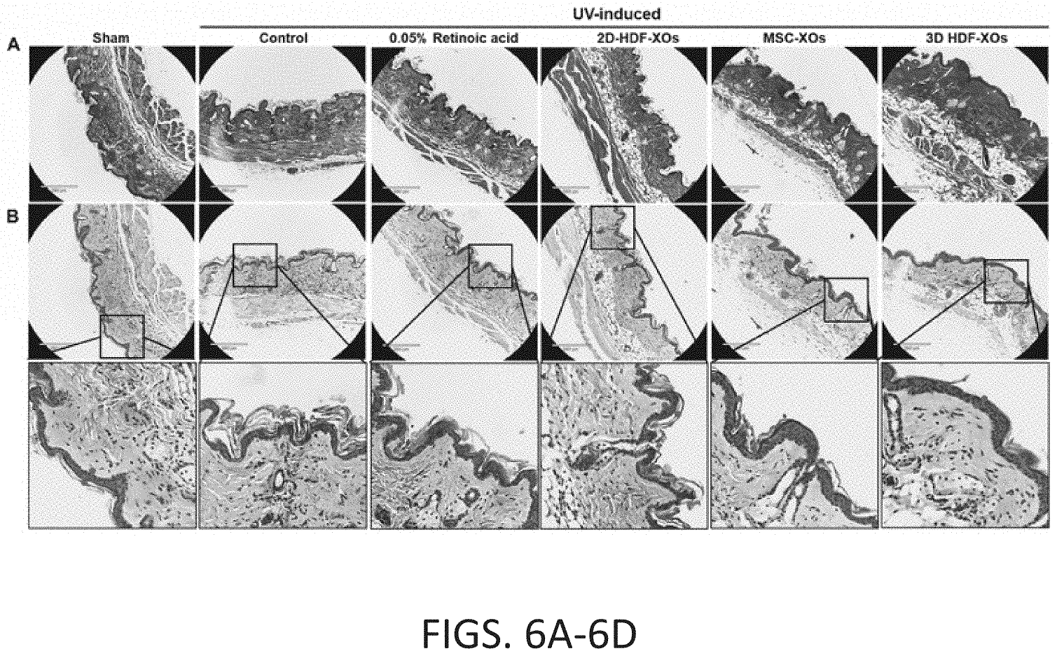

The effects of exosomes on wrinkle formations after UVB irradiation was investigated on the dorsal skin of mice. As shown in FIG. 5, there were almost no wrinkles in the sham group. Skin Replicas were imaged and analyzed to compare the number and thickness of wrinkles. In contrast, deep and wide wrinkles form in the control group due to UVB irradiation. With the treatment of exosomes by jet injection or 0.05% RA, the wrinkles in treated groups were more superficial and thinner. RA is the bioactive metabolite of vitamin A, and it has been proven efficacy in the treatment of photoaged skin and approved for clinical use. Usually, it will take two months (e.g., on mice) to see the effect of RA treatment since it is topical; the absorption and efficiency are quite limited. Exosomes were delivered directly into the dermis by the jet injector, which is much efficient than topical treatment. The single treatment started to show visible effect three weeks after treatment. Overall, the efficacy of 2D HDF-XOs was not comparable to RA, showing superficial, but lots of fine wrinkles. MSC-XOs showed a much better anti-wrinkle efficacy to RA, while 3D HDF-XOs provided the best skin treatment, leading to significantly thinner and more superficial wrinkles.

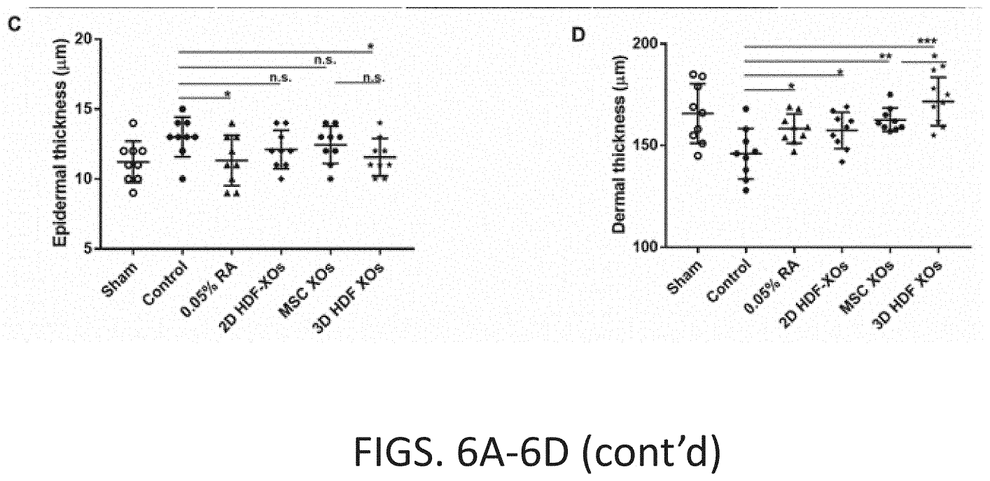

Skin histology elucidated the effects that exosomes had on the structural changes and the amount of collagen deposition in the dorsal skin (FIG. 6). Masson's trichrome staining showed the changes in the quantity of collagen in the dermis. The sham group displayed regularly arranged collagen fibers. Compared with the sham group, UV irradiation caused large amounts of abnormal, fragmented, and disorganized collagen fibers in the UV control group. Additionally, more inflammatory cells can be seen in the skin tissue of the UV control group than in other groups. Significant histological changes such as a woven stratum corneum and the disruption of collagen, were observed in the control group. All treated groups showed improvements in UV-induced damage to collagen fibers. Of all the treatments, 3D HDF-XOs resulted in the most abundant and dense collagen fibers, the most compact stratum corneum, and the thinnest epidermal layers compared to the control group.

### Example 6

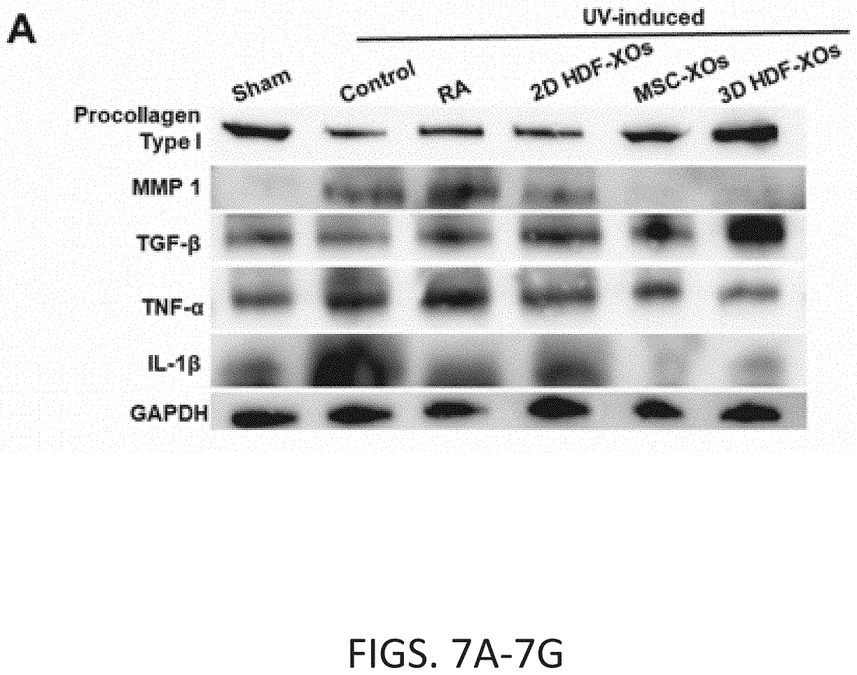

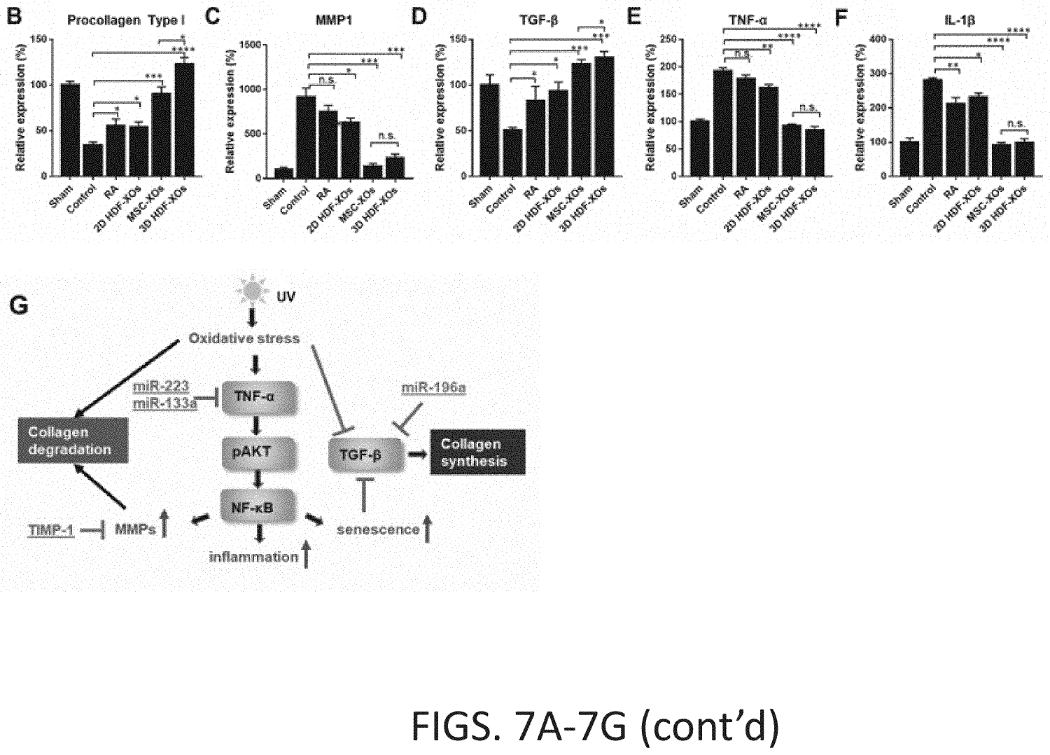

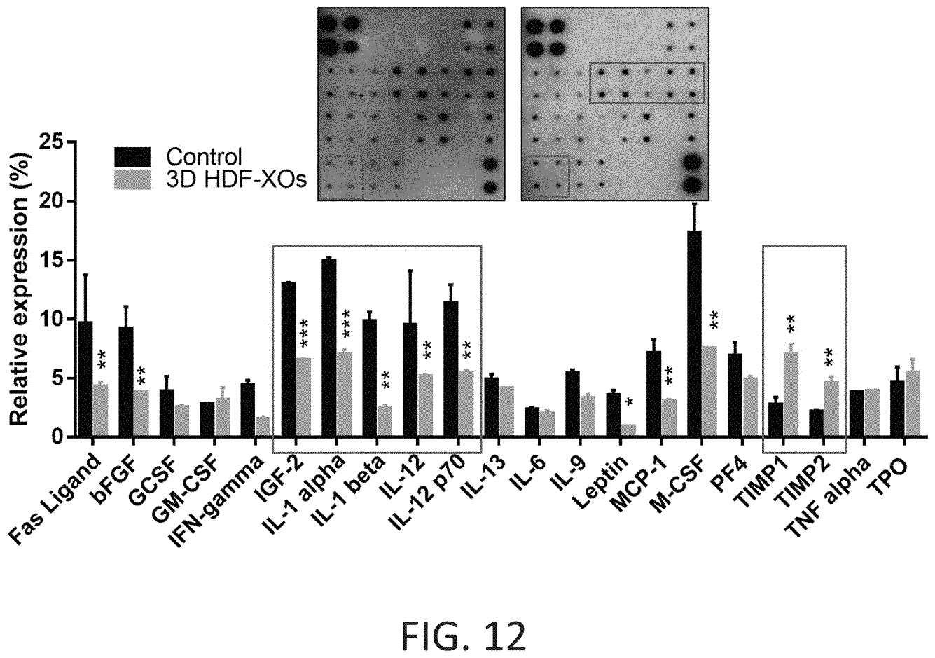

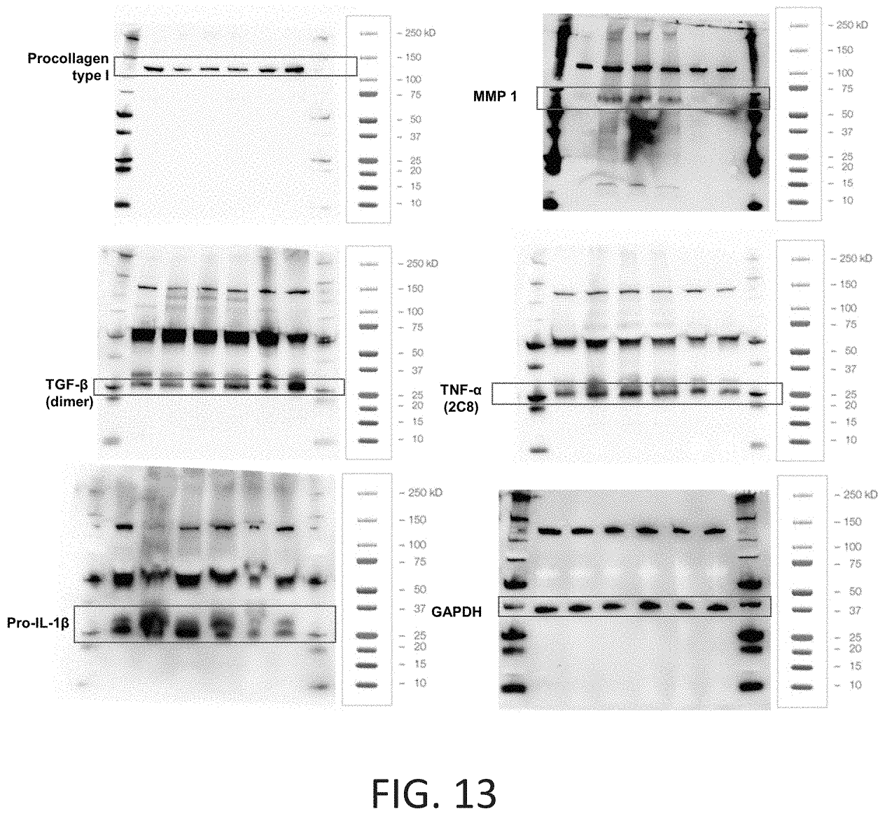

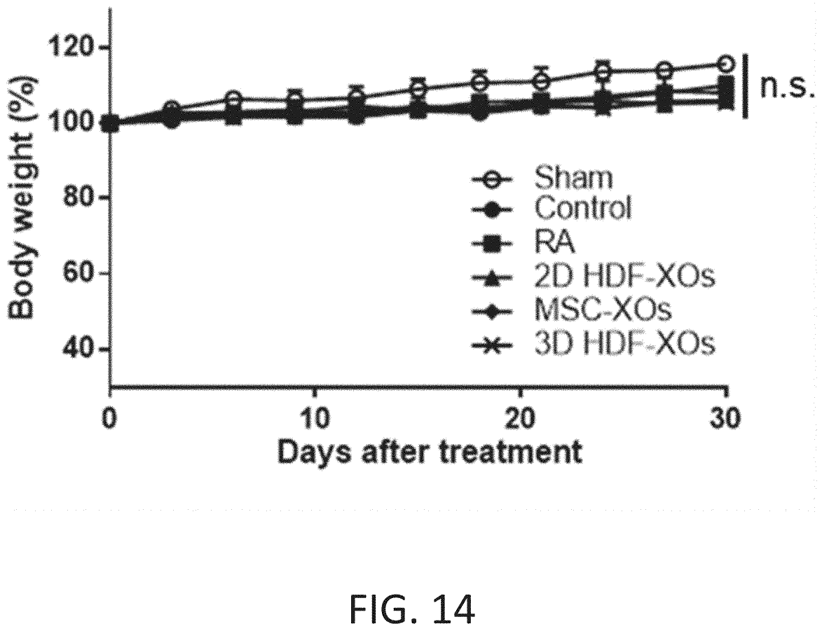

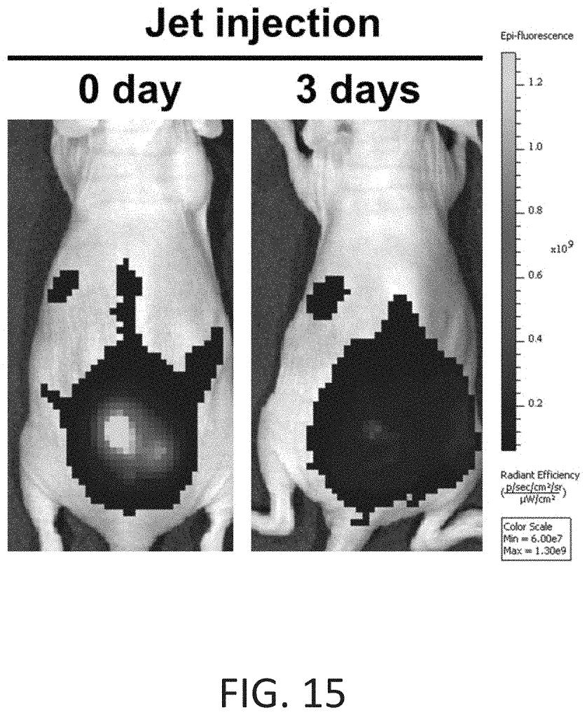

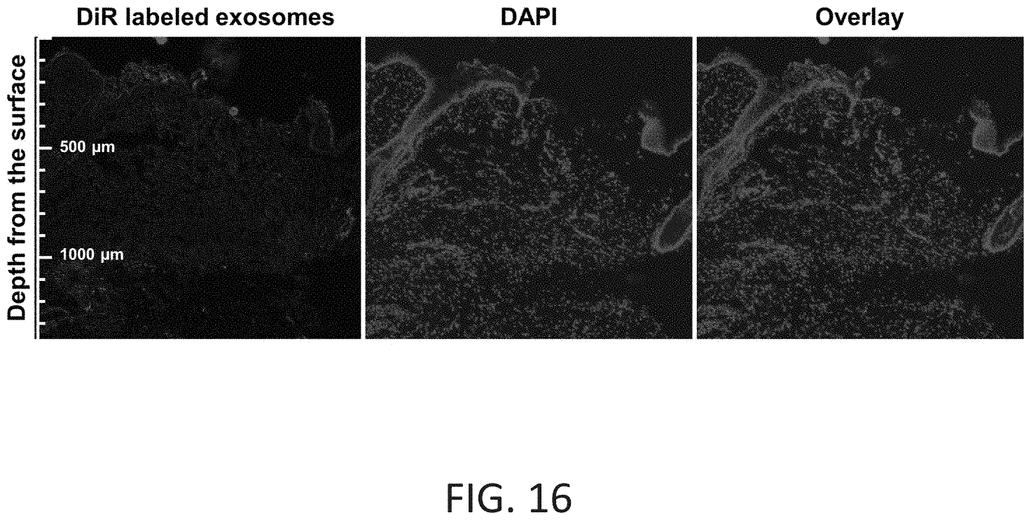

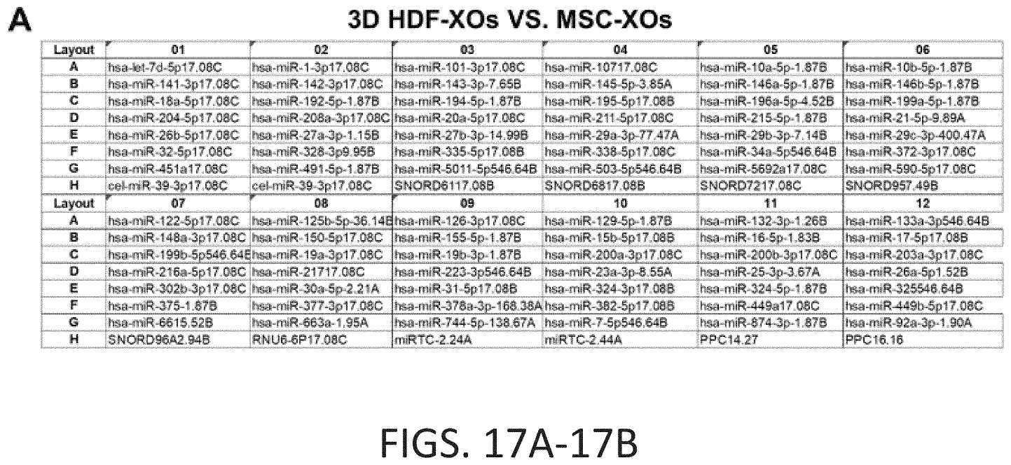

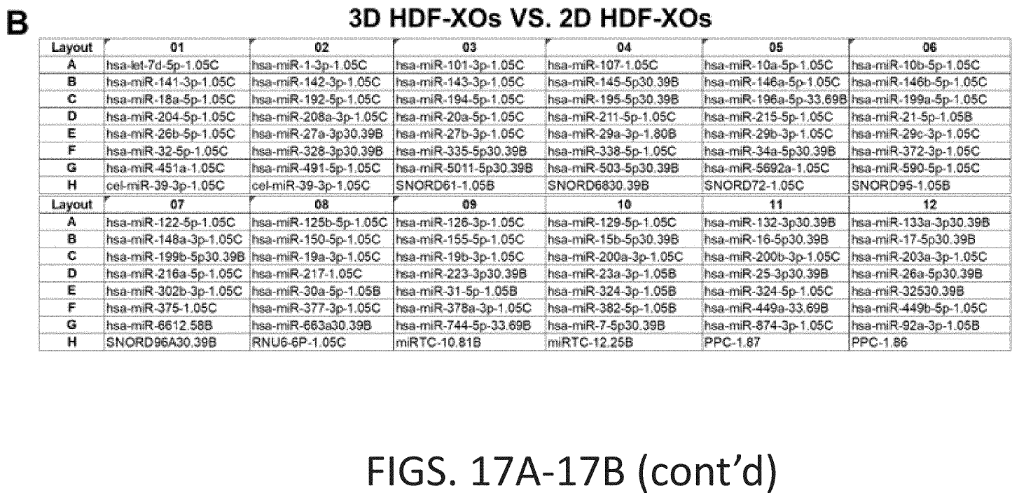

Effects of Exosomes on MMP-1 and Type I Procollagen Expression in Skin Tissue. To elucidate the molecular mechanisms that 3D HDF-XOs-driven amelioration of skin aging and collagen degradation in a UV-induced skin-aging model on nude mice, an apoptosis protein array was performed on skin samples after treatment. As shown in FIG. 12, the 3D HDF-XOs treatment group showed less apoptotic and inflammatory factors, such as Fas ligand, IFN-γ, and IL-1β, and higher expressions of TIMP-1 and TIMP-2. From these data, it is evident that the treatment efficacy of 3D HDF-XOs partially comes from reduced inflammation and the suppression of MMPs. Thus, type 1 procollagen, MMP-1, IL-1β and TGF-β expression in all groups was further investigated using western blotting (FIGS. 7A-7F).

Both UV-induced photoaging and intrinsic aging reduced procollagen type I synthesis by blocking the TGFβ signaling pathway and enhanced inflammatory, MMPs synthesis by upregulating TNF-α. The synthesis of procollagen was significantly enhanced and TGFβ signaling pathway was activated for all treated groups, especially for MSC-XOs and 3D HDF-XOs groups. The expression of procollagen type 1 and TGF-β in 3D HDF-XOs group were significantly higher than MSC-XOs. Meanwhile, the expression of MMP-1 was decreased to normal level in groups after treatment with MSC-XOs and 3D HDF XOs, compared with the control group. RA and 2D HDF-XOs showed a limited repairment and regulation capacity. The expression of IL-1β and TNF-α were drastically increased in the UVB-irradiated control group, which means a higher level of inflammation, MMPs and senescence. For MSC-XOs and 3D HDF-XOs, they both ameliorated the inflammation induced by UV irradiation and activated TGF-β.

MSC-XOs are well known for their ability to reduce inflammation, accelerate skin cell migration, improve angiogenesis and even ameliorate skin aging; however, results from the present disclosure demonstrate that 3D HDF-XOs exhibit a surprising and unexpected ability to regulate dermal fibroblasts to produce more procollagen and TIMP-1 to inhibit collagen degradation. The results described in FIG. 7G identify the possible mechanisms of 3D HDF-XOs in anti-skin aging. UV induced the enhanced oxidative stress in the skin, which activated the TNF-α and NF-κB signaling pathway, leading to the degradation of collagens and senescence consequently. Overall, results of the present discourse indicated that the 3D HDF-XOs group is the most efficacy in protecting skin from photoaging. For example, TIMP-1 and TGF-β, which are important in MMP suppression and regulating matrix synthesis, were upregulated, while TNF-α was downregulated.

What is claimed is:

1. A composition comprising a plurality of exosomes derived from human dermal fibroblast (HDF) spheroids, wherein the plurality of exosomes have at least one of:
(i) increased expression of Tissue Inhibitor of Metalloproteinases-1 (TIMP-1);
(ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or
(iii) decreased expression of at least one of the following miRNAs: hsa-miR-196a-5p and/or hsa-miR-744-5p.

2. The composition of claim 1, wherein the plurality of exosomes are derived from HDF spheroids cultured using three-dimensional (3D) cell culture.

3. The composition of claim 1, wherein the plurality of exosomes have at least one of (i)-(iii) as compared to a naturally occurring HDF-derived exosome.

4. The composition of claim 1, wherein the plurality of exosomes have at least one of (i)-(iii) as compared to an HDF-derived exosome cultured using two-dimensional (2D) cell culture.

5. A method of generating a plurality of exosomes capable of modulating at least one characteristic of skin tissue, the method comprising:
culturing human dermal fibroblast (HDF) spheroids using three-dimensional (3D) cell culture; and
isolating a plurality of exosomes from the HDF spheroids.

6. The method of claim 5, wherein the plurality of exosomes have at least one of:
(i) increased expression of Tissue Inhibitor of Metalloproteinases-1 (TIMP-1);
(ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or
(iii) decreased expression of at least one of the following miRNAs: hsa-miR-196a-5p and/or hsa-miR-744-5p.

7. The method of claim 6, wherein the plurality of exosomes have at least one of (i)-(iii) as compared to a naturally occurring HDF-derived exosome.

8. The method of claim 6, wherein the plurality of exosomes have at least one of (i)-(iii) as compared to an HDF-derived exosome cultured using two-dimensional (2D) cell culture.

10. A method of treating a skin condition or disease, the method comprising:
administering a plurality of exosomes derived from human dermal fibroblast (HDF) spheroids to a subject in need thereof;
wherein administering the plurality of exosomes modulates at least one characteristic of the subject's skin tissue.

11. The method of claim 10, wherein the plurality of exosomes have at least one of:
(i) increased expression of Tissue Inhibitor of Metalloproteinases-1 (TIMP-1);
(ii) increased expression of at least one of the following miRNAs: hsa-miR-133a-3p, hsa-miR-223-3p, hsa-5011-5p, hsa-miR-325, hsa-miR-199b-5p, and/or hsa-miR-34a-5p; and/or
(iii) decreased expression of at least one of the following miRNAs: hsa-miR-196a-5p and/or hsa-miR-744-5p.

12. The method of claim 10, wherein the plurality of exosomes are derived from HDF spheroids cultured using three-dimensional (3D) cell culture.

13. The method of claim 10, wherein the plurality of exosomes have at least one of (i)-(iii) as compared to a naturally occurring HDF-derived exosome.

14. The method of claim 10, wherein the plurality of exosomes have at least one of (i)-(iii) as compared to an HDF-derived exosome cultured using two-dimensional (2D) cell culture.

15. The method of claim 10, wherein the plurality of exosomes are administered to the subject's skin via injection, microinjection (microneedles), intradermal (ID) injection, subcutaneous (SC) injection, a non-invasive method, needle-free injection, or topical application.

16. The method of claim 10, wherein modulating at least one characteristic of the subject's skin tissue comprises at least one of:
(i) increasing expression of Transforming Growth Factor-β1 (TGF-β1);
(ii) increasing expression of procollagen type I;
(iii) decreasing expression of Tumor Necrosis Factor-α (TNF-α);
(iv) decreasing expression of Matrix Metallopeptidase 1 (MMP1);
(v) decreasing expression of Matrix Metallopeptidase 9 (MMP9); and/or
(vi) increased dermal collagen deposition.

17. The method of claim 16, wherein modulating at least one of (i)-(vi) treats the subject's skin condition or disease.

18. The method of claim 10, wherein the skin condition or disease comprises cutaneous aging.

19. The method of claim 10, wherein the skin condition or disease comprises cutaneous photoaging.

20. The method of claim 10, wherein the skin condition or disease comprises chronological aging.
